(19) United States
(12) Patent Application Publication
(10) Pub. No.: US 2020/0035363 A1
(43) Pub. Date: Jan. 30, 2020

(54) SYSTEM TO PREDICT HEALTH OUTCOMES

(71) Applicant: Siemens Healthcare GmbH, Erlangen (DE)

(72) Inventors: Sebastian Vogt, Monument, CO (US); Thomas Mertelmeier, Erlangen (DE)

(21) Appl. No.: 16/046,007

(22) Filed: Jul. 26, 2018

(51) Int. Cl. G16H 50/30; G16H 10/60; G16H 30/20; G06N 3/08

(52) U.S. Cl. CPC G16H 50/30; G06N 3/08; G16H 30/20; G16H 10/60

(57) ABSTRACT

A system and method includes acquisition of one or more images of each of a plurality of bodies, each of the images associated with an acquisition time, determination, for each body, of a future health status of the body, the future health status of the body being a health status of the body at a time after the acquisition time of the one or more images of the body, and training of an artificial neural network to output a predicted health status, the training based on the one or more images and determined future health status of each body.

### BACKGROUND

Medical imaging systems acquire images of internal patient volumes. A radiologist uses these images to identify and diagnose disease. For example, a radiologist identifies features within an image, such as lesions, masses and architectural distortions, and formulates a diagnosis based on the identified features. Current disease identification and diagnosis techniques therefore rely heavily on the skill and subjective judgment of the radiologist.

The efficacy of such techniques is further limited by a lack of relevant information, since only a few types of relevant features are known, and each type is associated with a small number of attributes (e.g., lesion size, tissue density, mass opacity, fuzziness). Also, at early stages of disease, images may seem “normal” and therefore abnormalities are not detected by even the most skilled radiologist. Moreover, radiologists possess a limited ability to analyze the complexity of patterns within radiographic images, either separately or in combination with additional patient data, such as patient history, genetic data, and other in-vivo and in-vitro data.

Incorrect diagnoses may result in negative health outcomes. In a false negative diagnosis, an image of a patient is acquired and a radiologist does not identify disease within the image. However, the patient develops a disease which could have been treated more effectively if it were diagnosed at the time of imaging. In a false positive diagnosis, the radiologist identifies disease when in fact no disease exists and the patient is subjected to needless additional testing and invasive treatment procedures.

A variety of semi-automatic and automatic methods have been developed to assist in the evaluation of medical images. Such methods include computer-aided detection and diagnosis algorithms which automatically locate and characterize visible lesions, and methods for automatically determining tissue density and morphological features. More recently, it has been proposed to train artificial neural networks using medical images which have been manually reviewed by an expert radiologist and labeled with a diagnosis. After deployment, during operation, such a trained network may receive a medical image and output automated findings. However, the training images must be accurately labeled by the expert radiologist, which might not be the case if no disease is detected yet still develops in the future. Moreover, the number of available labeled training images is limited.

What is needed are systems to efficiently correlate medical images with the likelihood of current or future disease, even if such disease is difficult to manually detect. Such systems may allow for early treatment, which is typically associated with the highest success and survival rates.

### BRIEF DESCRIPTION OF THE DRAWINGS

FIG. 1 is a block diagram of a system to train an artificial neural network to output health outcomes according to some embodiments;

FIG. 2 is a block diagram of a system deploying a trained artificial neural network to output health outcomes according to some embodiments;

FIG. 3 is a flow diagram of a process to train and deploy an artificial neural network to output health outcomes according to some embodiments;

FIG. 4 is a block diagram of a system to train an artificial neural network to output health outcomes according to some embodiments;

FIG. 5 is a block diagram of a system to train an artificial neural network to output health outcomes according to some embodiments;

FIG. 6 is a block diagram of a system deploying a trained artificial neural network to output a health status according to some embodiments;

FIG. 7 is a block diagram of a computing system to train an artificial neural network to output health outcomes according to some embodiments; and

FIG. 8 is a diagram of an imaging system deploying a trained artificial neural network to output a health status according to some embodiments.

### DETAILED DESCRIPTION

The following description is provided to enable any person in the art to make and use the described embodiments and sets forth the best mode contemplated for carrying out the described embodiments. Various modifications, however, will remain apparent to those in the art.

Generally, the embodiments described herein utilize artificial neural networks (ANNs) which are trained to correlate medical images with future health outcomes. Advantageously, embodiments are trained to correlate future health outcomes with medical images which might not depict features which are used by traditional techniques to predict future health outcomes. As will be described below, the training and deployment of an artificial neural network according to some embodiments leverages the fact that disease progression is a continuous process executing within a continuous system (i.e., the patient), and typically follows several transitory stages that tend to be similar for a given disease. By diagnosing disease earlier and more accurately than traditional methods, some embodiments facilitate early and effective intervention as well as avoidance of unnecessary or harmful intervention.

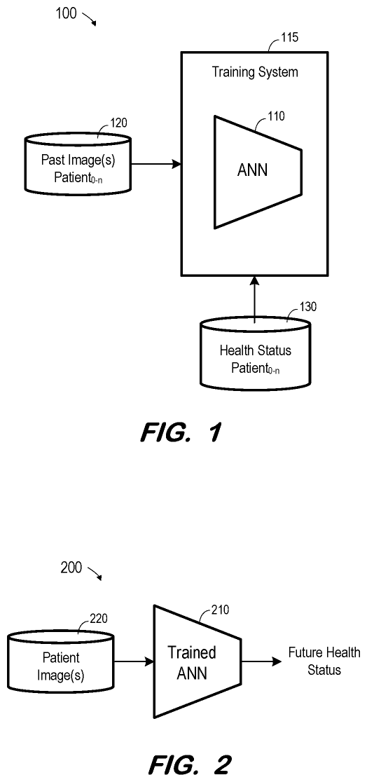

FIG. 1 illustrates system 100 to train an artificial neural network to predict health outcomes based on one or more images according to some embodiments. Artificial neural network 110 is trained using training system 115, past image data 120 and health status data 130. Past image data 120 may include one or more images of each of a plurality of patient bodies (patient₀₋ₙ), and health status data 130 may indicate a health status for each patient. Typically, data 120 and 130 comprise a large number of data sets (i.e., n is a large number).

Notably, past image data 120 and health status data 130 may be temporally distant. In other words, past image data 120 of a particular patient may have been acquired at a time before the health status of the particular patient, as reflected in health status data 130, was known. In one example, past image data 120 may include mammographic images from annual breast cancer screenings of a plurality of patients, where each image is at least one year old, while health status data 130 includes current breast cancer diagnoses of those patients. The current diagnoses may be acquired via analysis of current medical images by expert radiologists or by current knowledge as to whether the patient is actually suffering from (or perhaps died as a result of) the disease of interest, or from other medical tests (e.g., tissue samples from biopsy).

In some embodiments, past image data 120 of images depict one body region and health status data 130 is associated with disease of another body region. For example, the quality and quantity of belly fat, as determined by abdominal CT imaging, has been linked to the risk of developing a cardiovascular disease, and bone density/osteoporosis has been linked with chronic obstructive pulmonary disease.

Artificial neural network 110 may be trained to model the correlations between past image data 120 and health status data 130. As described with respect to FIG. 2, this model may then be used to predict health outcomes based on patient image data. Since training of network 110 in this manner may allow network 110 to learn subtle images features and their correlations with diseases that are not directly accessible to a human observer or a traditional diagnosis system, embodiments may therefore associate medical images with a diagnosis that traditional methods would only be capable of providing at a later time.

The images of past image data 120 may comprise any type of medical images that are or become known. The images may be two-dimensional and/or three-dimensional. Examples include but are not limited to X-ray images, computed tomography images, tomosynthesis images, magnetic resonance images, single-photon emission computed tomography images, and positron emission tomography images. Past image data 120 associated with a particular patient may comprise time-series data. Referring to the above example, past image data 120 may include, for each patient, mammographic images from two or more consecutive annual breast cancer screenings.

The future health status 130 associated with each patient health status may comprise any label. Examples include a binary value, such as 1 for a diagnosis indicating presence of disease and 0 for a diagnosis indicating no presence of disease. Future health status 130 may also comprise an indication of a disease stage such as an integer between 0 through 5, inclusive, or an indication of one or multiple of several diseases.

Broadly, artificial neural network 110 may comprise a network of neurons which receive input, change internal state according to that input, and produce output depending on the input and internal state. The output of certain neurons is connected to the input of other neurons to form a directed and weighted graph. The weights as well as the functions that compute the internal state can be modified by a training process based on ground truth data. Artificial neural network 110 may comprise any one or more types of artificial neural network that are or become known, including but not limited to convolutional neural networks, recurrent neural networks, long short-term memory networks, deep reservoir computing and deep echo state networks, deep belief networks, and deep stacking networks.

Training system 115 may comprise any system or systems for training an artificial neural network that are or become known. For example, training system 115 may employ supervised learning, unsupervised learning and/or reinforcement learning.

According to some embodiments, trained artificial neural network 110 implements a function. The function may be characterized as a set of parameter values associated with each network node. In one example, the function is represented by parameter values for kernels of a fully convolutional network. The function (e.g., embodied in parameter values of trained convolutional kernels) may be deployed as is known in the art to an external system such as system 200 of FIG. 2.

System 200 includes trained artificial neural network 210. Network 210 may be trained as described above with respect to network 100. Although depicted as an ANN, network 210 may comprise any type of processing system to implement the function resulting from the training of network 110 of FIG. 1.

In operation, one or more images 220 of a patient are acquired and are input to trained artificial neural network 210. The number and type of the images 220 correspond to the number and type of images per training sample used to train network 110. For example, if network 110 was trained based on a single three-dimension computed tomography image of each patient, input image 220 consists of a single three-dimension computed tomography image of a patient. Network 210 then operates to output a future health status based on the input image.

Embodiments may therefore learn and recognize disease patterns in medical images at early stages of disease, to an extent which exceeds capabilities of a skilled radiologist. These disease patterns are not limited to specific details and features found in screening images, such as lesions and masses, but can encompass otherwise indiscernible patterns within the images that have not yet been correlated with diseases by traditional reading methods. In a case that a network is trained based on time-series images, the patterns may include changes over time that are either undetectable by traditional reading methods or appear transcendent thereto.

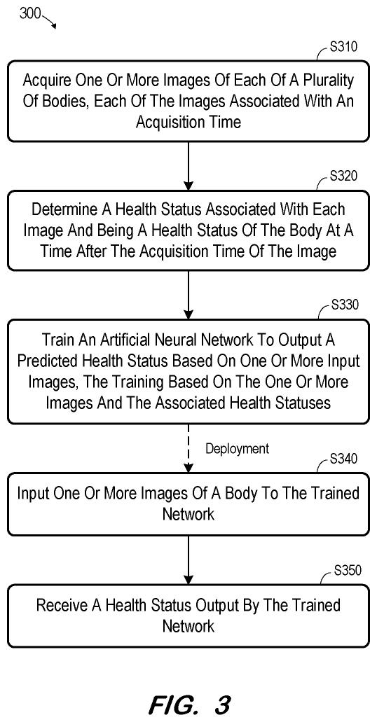

FIG. 3 is a flow diagram of process 300 depicted in FIGS. 1 and 2 according to some embodiments. Process 300 and the other processes described herein may be performed using any suitable combination of hardware and software. Software program code embodying these processes may be stored by any non-transitory tangible medium, including a fixed disk, a volatile or non-volatile random access memory, a floppy disk, a CD, a DVD, a Flash drive, or a magnetic tape. Embodiments are not limited to the examples described below.

Initially, at S310, one or more images of each of a plurality of bodies is acquired. Each of the images associated with an acquisition time. The one or more images may be acquired from one or more public or private repositories of past medical images. At S320, a health status is determined for each image. For each image of a body, the health status is a health status of the body at some time after the acquisition time of the image. For example, the acquired images may comprise mammographic images of a plurality of patients which were generated two years ago, and the health status determined at S320 may be a current health status of each patient.

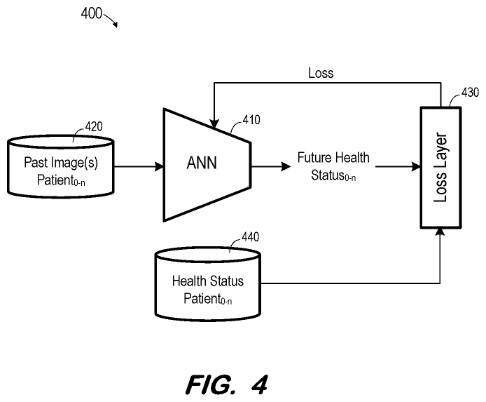

Next, and as described with respect to FIG. 1, an artificial neural network is trained based on the one or more acquired images and the associated health statuses. The network is trained to output a predicted health status based on one or more input images. FIG. 4 illustrates training architecture 400 according to some embodiments. Embodiments are not limited to the type of training depicted in FIG. 4.

During training, network 410 receives one or more past images 420 of patient₀₋ₙ. Based on its initial configuration and design, network 410 outputs a future health status for each patient based on the one or more input images of the patient. Loss layer component 430 determines a loss by comparing each output future health status₀₋ₙ to the actual health status 440 of each of patient₀₋ₙ. For example, network 410 generates future health status₉ based on one or more past images 420 of patient₉. Loss layer component 430 compares generated future health status₉ to actual health status 440 of patient₉ to determine a loss. The foregoing is performed for each patient₀₋ₙ to determine a total loss. The loss may comprise an L1 loss, and L2 loss, or any other suitable measure of total loss.

The total loss is back-propagated from loss layer component 430 to network 410, which changes its internal weights in response as is known in the art. The process repeats until it is determined that the total loss has reached an acceptable level or training otherwise terminates. At termination, network 410 may be considered trained.

Returning to process 300, the trained network is deployed after S330. Deployment may comprise determining the function represented by the trained network and implementing the function in another computing system according to techniques that are or become known.

After deployment, one or more input images of a body are input to the trained network at S340. Next, at S350, the trained network operates to apply its learned function to the input images and output a predicted health status. According to some embodiments, the predicted health status comprises a likelihood of each of one or more possible classifications.

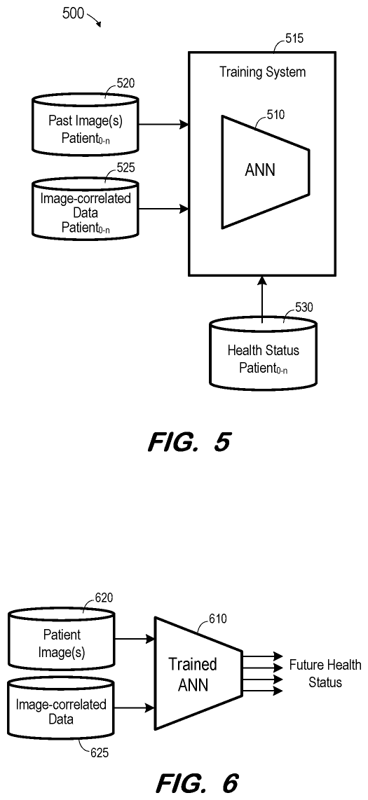

FIG. 5 illustrates architecture 500 according to some embodiments. Architecture 500 is similar to architecture 100 except for the addition of training data 525. Training data 525 may comprise data correlated with the images of each patient₀₋ₙ. This data may comprise any in-vitro or in-vivo data corresponding to a patient or to an image, such as but not limited to age, family history, blood values at imaging time, and DICOM image header information. Including data 525 in the training process may improve the predictive ability of the learned function.

FIG. 6 illustrates example deployment of network 610 which was trained based on the training data shown in FIG. 5. As shown, network 610 receives one or more images 620 of a patient and image-correlated data 625 and outputs a future health status based thereon. The type of image-correlated data 625 depends upon the type of data 525 used to train network 610. Network 625 is configured and trained to predict likelihoods (or probabilities) associated with each of four diseases in parallel, although embodiments are not limited to any particular number of diseases.

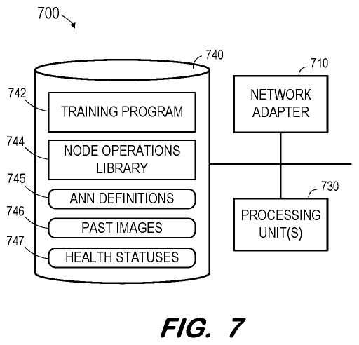

FIG. 7 illustrates computing system 700 according to some embodiments. System 700 may comprise a computing system to facilitate the design and training of artificial neural networks as is known in the art. Computing system 700 may comprise a standalone system, or one or more elements of computing system 700 may be network or cloud-located.

System 700 includes network adapter 710 to communicate with external devices via a network connection. Processing unit(s) 730 may comprise one or more processors, processor cores, or other processing units to execute processor-executable process steps. In this regard, storage system 740, which may comprise one or more memory devices (e.g., a hard disk drive, a solid-state drive), stores processor-executable process steps of training program 742 which may be executed by processing unit(s) 730 to train an artificial neural network as described herein.

Training program 742 may utilize node operations library 744, which includes code to execute various operations associated with network nodes as defined in artificial neural network definitions 745. According to some embodiments, computing system 700 provides interfaces and development software (not shown) to enable development of training program 742 and generation of definitions 745. Storage device 740 also includes past images 746 and health statuses 747, which may be used by training program as described above to train a network defined within definitions 745.

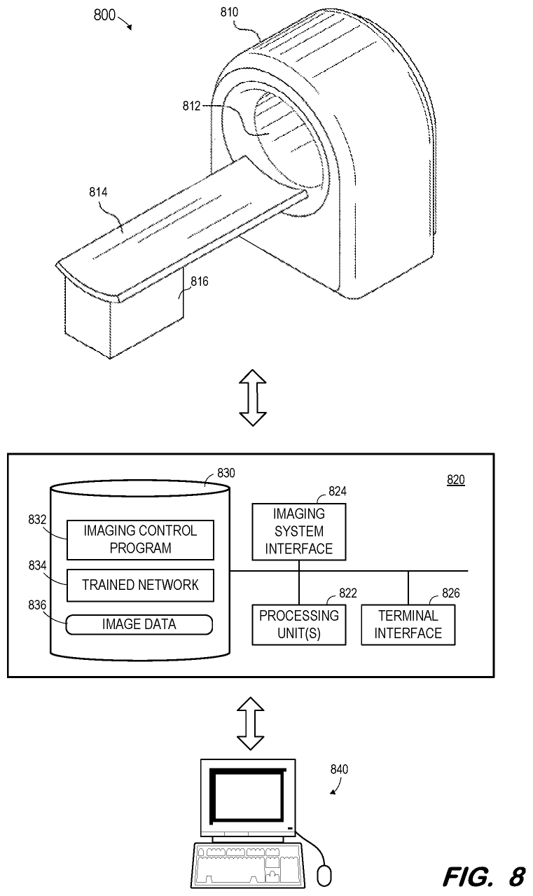

FIG. 8 illustrates medical imaging system 800 in which a trained network may be deployed according to some embodiments. Generally, imaging system 800 may acquire one or more images of a patient and input the images to a trained network as described herein in order to generate a predicted health status. Embodiments are not limited to system 800 or to any imaging system. For example, a separate computing system may receive images from an imaging system or from a picture archiving and communications system and input those images to a trained network implemented by the separate computing system in order to generate a predicted health status.

System 800 includes gantry 810 defining bore 812. As is known in the art, gantry 810 houses CT imaging components for acquiring CT image data. The CT imaging components may include one or more x-ray tubes and one or more corresponding x-ray detectors.

Bed 814 and base 816 are operable to move a patient lying on bed 814 into and out of bore 812. Movement of a patient into and out of bore 812 may allow scanning of the patient using the CT imaging elements of gantry 810.

Control system 820 may comprise any general-purpose or dedicated computing system. Accordingly, control system 820 includes one or more processing units 822 configured to execute processor-executable program code to cause system 820 to operate as described herein, and storage device 830 for storing the program code. Storage device 830 may comprise one or more fixed disks, solid-state random access memory, and/or removable media (e.g., a thumb drive) mounted in a corresponding interface (e.g., a USB port).

Storage device 830 stores program code of imaging control program 832. One or more processing units 822 may execute imaging control program 832 to, in conjunction with imaging system interface 824, cause a radiation source within gantry 810 to emit radiation toward a body within bore 812 from different projection angles, and to control a corresponding detector to acquire two-dimensional CT data. The acquired data may be stored in memory 830 as image data 836.

One or more processing units 822 may also execute code implementing trained network 834. The code may be exported by system 700 after training of a network. The code may be executed to receive image data from image data 836 and to generate a predicted health status based thereon.

An acquired image and predicted health status may be transmitted to terminal 840 via terminal interface 826. Terminal 840 may comprise a display device and an input device coupled to system 840. In some embodiments, terminal 840 is a separate computing device such as, but not limited to, a desktop computer, a laptop computer, a tablet computer, and a smartphone.

Those in the art will appreciate that various adaptations and modifications of the above-described embodiments can be configured without departing from the claims. Therefore, it is to be understood that the claims may be practiced other than as specifically described herein.

What is claimed is:

1. A computing system comprising:
a storage system;
one or more processors to execute processor-executable process steps stored on the storage system to cause the computing system to:
acquire one or more images of each of a plurality of bodies, each of the images associated with an acquisition time;
for each body, determine a future health status of the body, the future health status of the body being a health status of the body at a time after the acquisition time of the one or more images of the body;
train an artificial neural network to output a predicted health status, the training based on the one or more images and a determined future health status of each body; and
use the trained artificial neural network to output a first predicted health status of a first body based on a first one or more images of the first body.

2. A computing system according to claim 1, the one or more processors to further execute processor-executable process steps stored on the storage system to cause the computing system to output parameter values of trained convolutional kernels of the trained artificial neural network to a second computing system.

3. A computing system according to claim 1, wherein acquisition of the one or more images of each of the plurality of bodies comprises acquisition of in vivo or other data indicating at least one of: age, family history, blood values, and DICOM image header information of each of the plurality of bodies, and wherein the training is based on the one or more images, determined future health status, and the in vivo or other data of each body.

4. A computing system according to claim 1, wherein the future health status of each body is either a first value or a second value, and wherein the predicted health status is a likelihood of the first value and a likelihood of the second value.

5. A computing system according to claim 1, wherein the future health status of each body is one of a plurality of values, and wherein the predicted health status comprises likelihoods of each of the plurality of values.

6. A computing system according to claim 1, wherein the one or more images of each body comprises time-series image data.

7. A computing system according to claim 6, wherein acquisition of the one or more images of each of the plurality of bodies comprises acquisition of in vivo or other data indicating at least one of: age, family history, blood values, and DICOM image header information of each of the plurality of bodies, and wherein the training is based on the one or more images, determined future health status, and the in vivo or other data of each body.

8. A computing system according to claim 1, wherein each of the one or more images depicts a first body region and not a second body region, and wherein the determined future health statuses are associated with the second body region.

9. A method comprising:
acquiring one or more images of each of a plurality of bodies, each of the images associated with an acquisition time;
for each body, determining a future health status of the body, the future health status of the body being a health status of the body at a time after the acquisition time of the one or more images of the body; and
training an artificial neural network to output a predicted health status, the training based on the one or more images and determined future health status of each body.

10. A method according to claim 9, further comprising:
outputting parameter values of trained convolutional kernels of the trained artificial neural network.

11. A method according to claim 9, wherein acquiring the one or more images of each of the plurality of bodies comprises acquiring in vivo or other data indicating at least one of: age, family history, blood values, and DICOM image header information of each of the plurality of bodies, and wherein the training is based on the one or more images, determined future health status, and the in vivo or other data of each body.

12. A method according to claim 9, wherein the future health status of each body is either a first value or a second value, and wherein the predicted health status is a likelihood of the first value and a likelihood of the second value.

13. A method according to claim 9, wherein the future health status of each body is one of a plurality of values, and wherein the predicted health status comprises likelihoods of each of the plurality of values.

14. A method according to claim 9, wherein the one or more images of each body comprises time-series image data.

15. A method according to claim 14, wherein acquiring the one or more images of each of the plurality of bodies comprises acquiring other data indicating at least one of: age, family history, blood values, and DICOM image header information of each of the plurality of bodies, and wherein the training is based on the one or more images, determined future health status, and the other data of each body.

16. A method according to claim 9, wherein each of the one or more images depicts a first body region and not a second body region, and wherein the determined future health statuses are associated with the second body region.

17. A system comprising:
an artificial neural network;
stored data comprising one or more images of each of a plurality of bodies, each of the images associated with an acquisition time, and each of the one or more images associated with a body being also associated with a future health status of the body, the future health status being a health status of the body at a time after the acquisition times of the one or more images of the body; and
a training architecture to train the artificial neural network to output a predicted health status, the training based on the one or more images and determined future health status of each body.

18. A system according to claim 17, each of the one or more images of a body associated with other data indicating at least one of: age, family history, blood values, and DICOM image header information of the body, and wherein the training is based on the one or more images, determined future health status, and the other data of each body.

19. A system according to claim 17, wherein the future health status of each body is one of a plurality of values, and wherein the predicted health status comprises likelihoods of each of the plurality of values.

20. A system according to claim 17, wherein each of the one or more images depicts a first body region and not a second body region, and wherein the determined future health statuses are associated with the second body region.
